(19) United States
(12) Patent Application Publication
(10) Pub. No.: US 2026/0128173 A1
(43) Pub. Date: May 7, 2026

(54) Service to Automate the Risk Calculation of Genetic Disorders

(71) Applicants: Ricardo Jorge Fonseca Tavares Godinho Pais, Manchester (GB); Markella Andrea Mikkelsen, Manchester (GB)

(72) Inventors: Ricardo Jorge Fonseca Tavares Godinho Pais, Manchester (GB); Markella Andrea Mikkelsen, Manchester (GB)

(21) Appl. No.: 18/938,046

(22) Filed: Nov. 5, 2024

(51) Int. Cl. G16H 50/30; G16B 20/20; G16B 40/20; G16B 50/10; G16H 10/60; G16H 70/60

(52) U.S. Cl. CPC G16H 50/30; G16B 20/20; G16B 40/20; G16B 50/10; G16H 10/60; G16H 70/60

(57) ABSTRACT

The invention provides a method for the automated calculation of the reproductive risk of genetic disorders from Next Generation Sequencing (NGS) data of male and female subjects. The method includes (i) online data collection from male and female subjects; (ii) processing raw sequencing data to detect genetic variants; (iii) assessing variant pathogenicity using a scoring metric that comprises multiple types of supporting evidence; (iv) text mining of male and female family history and phenotype description; (v) association of genetic disorders to the identified pathogenic variants based on a comprehensive database with automated updating functionalities; (vi) calculation of the reproductive risks using data from male and female subjects; (vii) digital report generation. The method enables efficient and accurate identification of pathogenic variants in a wide range of gene-disease associations, which can be scaled in a computer system.

### FIELD OF THE INVENTION

The present invention belongs to the field of computational methods for identifying pathogenicity in genetic variants and its utility in clinical genetics.

### BACKGROUND TO THE INVENTION

The invention (“A Service to Automate the Risk Calculation of Genetic Disorders”, referred to thereafter as the “Service”) addresses a major healthcare challenge. According to the W.H.O., one in 100 children is born with a genetic disorder, which can be both life-limiting and life-threatening. There are over 7,000 known genetic disorders and currently 300 million people are affected by genetic disorders worldwide¹. The cost of treating genetic disease reached almost $1 trillion in the US in 2019². Additionally, caring for a child with a life-limiting genetic disease comes with a financial and societal burden to the families involved, where the care costs can reach up to $3 million per individual¹⁻⁴.

Screening for genetic disorders at preconception has been suggested as a viable mitigation strategy for life-limiting genetic disorders⁵. Understanding reproductive risk early, ideally at preconception stage, empowers prospective parents to adopt a proactive approach in managing that risk, potentially leading to a long-term reduction of the healthcare burden⁵. However, genomic testing is currently focused on solving diagnostic cases, rather than prognostication, due to the technological constraints detailed below. As a consequence, the implementation of large-scale preventive screening programs, inclusive of ethnically diverse populations, who could benefit from access to genetic testing remain financially and technically unviable.

The current state-of-the-art in genomic testing in a diagnostic setting is Next Generation Sequencing (NGS) technologies^{6,7}. The advantage of NGS as a high-throughput DNA technology is that it enables the screening of several thousand genes in a single test and therefore has the potential to be scalable^{8,9}. However, interpretation of NGS data for a clinical purpose is complex, labor-intensive and relies on highly-skilled individuals⁷.

The present invention centers on delivering a cost-effective, end-to-end computational system that uses scalable functionalities to enable millions to receive accurate genetic testing faster. Our solution addresses the following limitations of the current workflows:

1) Diagnosis vs prevention. Current approaches focus on executing confirmatory diagnosis on symptomatic individuals suspected to have a genetic disease using genome-wide studies⁶. There are few initiatives to extend this technology to a large-scale preventive strategy in terms of determining reproductive risk⁸. The latter tend to focus on a limited gene panel of the most common genetic diseases and usually test prospective parents sequentially. Therefore, the approach is limited with a gap of several thousand untested genes which potentially constitute reproductive risk. Furthermore, the strategy of testing the male and female parents sequentially creates unnecessary delays and limits the testing of the second parent to one or few gene targets. Currently, there is no scalable software solution designed for systematic calculation of reproductive risk for exome- or genome-wide screening. This is a limitation in implementing a preventive strategy.

2) Reliance on highly specialized labor prevents scalability. NGS data-processing frameworks are highly dependent on bioinformaticians for running multiple software tools and visually checking the data quality and file management⁷. This constitutes a potential bottleneck in scaling the service. Downstream analysis of data and selection of pathogenic genetic variants requires the input of a specialized clinical scientist trained in genomics. This is a case-by-case labor-intensive approach. These specialists perform the task of selecting reportable pathogenic candidate variants by using software to filter multiple data features and visualize supporting evidence¹⁰. The selection has to follow complex and extensive American College of Medical Genetics and Genomics (ACMG) guidelines^{5,11,12}. This constitutes another bottleneck in scaling the service. Software aiding genomic variant analysis and interpretation has resulted in improved solutions for gathering relevant data and facilitating data visualization. However, these software tools have not resulted in massively scalable genomic solutions due to dependence on manual steps.

3) High computational requirements that limit the scalability of services. Currently available software tools for Variant Calling File (VCF) annotation, such as GATK, ANNOVAR and others, are computationally demanding^{10,13}. VCF Annotations can take up to several hours depending on the number of variants, available computational resources on CPU, memory and disk space. This is because annotation tools are designed to be generic and execute several hundred standard feature annotations per variant in text files using multiple database accesses. The outcomes often lead to multiple VCFs containing redundant and unnecessary data.

### SUMMARY OF THE INVENTION

The invention is a process framework to deliver a cost-effective, end-to-end system that uses computationally scalable functionalities. These functionalities automate the processing, assessment and interpretation of NGS data with the purpose of identifying pathogenic or likely/pathogenic genetic variants in prospective parents (or egg/sperm providers) that can potentially result in the birth of a future child affected by a life-limiting genetic disease. The application of the invention enables the automated and scalable screening of over 7,000 genetic disorders in a single test, addressing limitations 1-3 highlighted in the background section.

The invention further enables a substantial improvement of reporting times and accuracy of candidate variant selection. This is achieved by removing the human intervention bottlenecks (limitation 2 in the Background section), human bias and error during the data analysis stage. Current implementation of the “Service” in the system enables a turnover of the entire process in the range of 2˜3 hours with a 97% accuracy.

The invention improves the NGS technology capacity for early identification of the risk of passing on life-limiting inherited diseases and enables more effective mitigation strategies and family planning.

The invention provides a new and compact database (MgenDA) specifically designed for the “Service” with a curated, up-to-date gene-disease catalog (FIG. 2). Auto-updating of MgenDA is integrated into the invention to periodically conduct automated online searches to databases and detect updates on main database sources. The auto-update of MgenDA also includes an automated search for relevant scientific publications using a trained Artificial Neural Network (ANN) in order to detect new variants or new gene-disease associations.

The invention performs automated matching between the subject's provided family history and genes with known association with the phenotypic features provided (“phenomatching”). This is achieved by keyword matching of the input text containing the disease description and phenotypic features provided with a library containing a glossary of disease features and names in the MgenDA database. The system computes a keyword matching score for each disease selected. For disease feature matching, the system relies on the application of NLP algorithms with augmented synonym databases, combined with fuzzy string patterning matching algorithms for optimizing the detection of true positives and minimizing false positives. This feature enables the extraction of a subset of genes associated with the matched phenotype for automated querying of the MgenDA database.

The “phenomatching” enables the inclusion, in the analysis process, of genes relevant to the subject's family history for Autosomal Dominant, Autosomal Recessive, X-linked and Y-linked diseases.

The invention implements modifications to the standard human NGS bioinformatics pipeline practices (secondary analysis) to enable automation and scalability, addressing limitations 2 and 3 in the Background section.

In one aspect, assessment of the sequencing QC is performed by the application of rule-based filters that apply default cut-off values of quality, addressing limitation 2 in the Background section.

In another aspect, the invention implements a reduction in the number of redundant annotations from databases which improves computational power and disk space, addressing limitation 3 in the Background section.

In another aspect, an online FASTQ file-uploading system and storage into BAM raw data with the annotated variant calling results saved into SQL databases/JSON files is part of the “Service”. In comparison with standard clinical bioinformatics practices, this enables: 1) optimization of data storage; 2) Automation of data storage and backup; 3) automation of re-processing and analysis; 4) Faster parsing of downstream analysis as this type of objects are natively optimized in most programming languages. All of the above address limitation 3 in the Background section.

The invention performs a systematic collection and evaluation of relevant annotated information from MgenDA to automate the identification of pathogenic variants. The invention applies automated classification of pathogenic/benign evidence using the ACMG criteria (ref) (e.g. PVS1, PS1, PM2, etc.) on the collected information. NLP algorithms and multiple pattern-matching algorithms are applied for the cases that require keyword matching on annotated information. Using the inferred evidence, the system automatically assigns one of the ACMG criteria to the variant to aid classification of the particular variant by applying the recommended published rules.

To enable the automated identification of pathogenic variants, the invention applies a novel evidence-based scoring system, the MolMart Integrative Ranking Score (MIRS). This is a quantitative metric that weights multiple sources of information used in genomics analysis to evaluate and decide on the pathogenicity potential of a variant.

During the “Service”, the computed MIRS values are systematically used for variant selection by applying a cut-off value to remove variants that are not pathogenic. The invention includes the application of machine learning techniques to estimate optimal cut-off values on large datasets, including artificial and real clinical data. This achieves high gains in performance (sensitivity and specificity) when compared with the performances of other commonly-used predictive tools (FIG. 7).

The invention performs systematic pairing of genomic data from one male and one female subject. For Autosomal Recessive mode of inheritance, the “Service” identifies co-occurrence of pathogenic variants in the paired data at the gene level to enable the identification of heterozygous (carrier) status. The system automatically computes the combined reproductive risk for the associated disease by applying Mendelian law of inheritance.

In another aspect, for Autosomal Recessive mode of inheritance after performing pairing of genomic data from one male and one female subject, the “Service” identifies co-occurrence of likely pathogenic variants in the paired data at the gene level to enable the identification of heterozygous (carrier) status. The system automatically computes the combined reproductive risk for the associated disease by applying Mendelian law of inheritance.

In another aspect, for Autosomal Recessive mode of inheritance after performing pairing of genomic data from one male and one female subject, the “Service” identifies occurrence of 2 different pathogenic variants on the same gene in the male and female subject to enable the identification of heterozygous (carrier) status and the designation of compound heterozygosity in the event of inheritance of those pathogenic variants from the male and female subjects.

In another aspect, for Autosomal Recessive mode of inheritance after performing pairing of genomic data from one male and one female subject, the “Service” identifies occurrence of 2 different likely pathogenic variants on the same gene in the male and female subject to enable the identification of heterozygous (carrier) status and the designation of compound heterozygosity in the event of inheritance of those likely pathogenic variants from the male and female subjects.

In another aspect, the invention identifies pathogenic variants(s) on the X chromosome(s) on genomic data from one male and one female subject, irrespective of co-occurrence of gene variant(s). In this case the reproductive risk is calculated based on the gender of the resulting embryo.

In another aspect, the invention identifies likely pathogenic variants(s) on the X chromosome(s) on genomic data from one male and one female subject, irrespective of co-occurrence of gene variant(s). In this case the reproductive risk is calculated based on the gender of the resulting embryo.

In another aspect, the invention identifies pathogenic variants for Autosomal Dominant disease on genomic data from one male and one female subject, separately and irrespective of co-occurrence of gene variant(s). A reproductive risk is given for Autosomal Dominant disease. The result is given in the context of the male or female subject phenotype, as appropriate.

In another aspect, the invention identifies likely pathogenic variants for Autosomal Dominant disease on genomic data from one male and one female subject separately and irrespective of co-occurrence of gene variant(s). A reproductive risk is given for Autosomal Dominant disease. The result is given in the context of the male or female subject phenotype, as appropriate.

In another aspect, the invention identifies pathogenic variants on the Y chromosome from genomic data of the male subject. A reproductive risk is calculated based on the gender of the resulting embryo.

In another aspect, the invention identifies likely pathogenic variants on the Y chromosome from genomic data of the male subject. A reproductive risk is calculated based on the gender of the resulting embryo.

The invention enables an automated reporting system that populates a report in Portable Document Format (PDF) or other digital format containing the identified pathogenic and/or likely pathogenic variants, reproductive risks and associated diseases.

The invention includes a web-based portal with a user interface for clients to login and access the generated case reports. The portal utilizes a Large Language Model (LLM), through a plugin, with a bot interface and a dialog box that mediates assistance for clinical data collection and basic interpretation of report information. This feature increases efficiency in human resources ensuring end-to-end scalability of the “Service”, thus addressing limitation 2 in the Background section.

### UTILITY OF THE INVENTION

1 The purpose of the invention is to calculate the reproductive risk for a male and a female partner (or an egg and a sperm provider) at the preconception stage and to provide a clinical report outlining the reproductive risk and the genetic variant(s) involved, as well as tailored recommendations and actionable solutions.
2 In another aspect, the purpose of the invention is to calculate the reproductive risk for a male and a female partner (or an egg and a sperm provider) at the early prenatal stage and to provide a clinical report outlining the reproductive risk and the genetic variant(s) involved, as well as tailored recommendations and actionable solutions.
3 In another aspect, the purpose of the invention is to calculate the individual reproductive risk for one egg donor and several sperm donors at the preconception stage and to provide a ranked report with lowest-to-highest ranked reproductive risk matches and the genetic variant(s) involved, as well as tailored recommendations and actionable solutions.
4 In another aspect, the purpose of the invention is to calculate the individual reproductive risk for one sperm donor and several egg donors at the preconception stage and to provide a ranked report with lowest-to-highest ranked reproductive risk matches and the genetic variant(s) involved, as well as tailored recommendations and actionable solutions.
5 In another aspect, the purpose of the invention is to identify and report the carrier status of a single individual, to provide the genetic variant(s) involved and to provide tailored recommendations and actionable solutions for that carrier.
6 In another aspect, the purpose of the invention is to identify carrier status of a genetic variant associated with an Autosomal Dominant disease; to identify the predisposition or elevated risk associated with the particular genetic variant; to provide tailored recommendations and actionable solutions to the carrier for the specific variant.
7 The invention applies all the above principles and can perform systematic pairing of genomic data between different male and female subject combinations and calculates the reproductive risk for each one of the combinations separately.
8 The invention applies all the above principles and embodiments and recalculates the respective reproductive risk for paired genomic data from male and female subjects in light of newly published information relating to updated status of: pathogenicity of a genetic variant; likely pathogenicity of a genetic variant; change of status of a Variant of Uncertain Significance (VUS) to pathogenic or likely pathogenic; and change of status of VUS to benign/likely benign.

All the above intended purposes of the invention are not limited to any particular geographical location or ethnic group or any one type of NGS genomic data.

### BRIEF DESCRIPTION OF THE FIGURES

FIG. 1. Overview of the whole system of the “Service”. The diagram shows all parts of the system that provide the automation of the service. Human intervention inputs are highlighted with gray filled oval shapes, rectangles represent the main processes and cylinders represent database structures. Male and female symbols indicate data transfers for male and female subjects, respectively. The cloud shape indicates cloud-based data storage. Dashed squares indicate the main core processes of the system, referred to as modules.

FIG. 2. Diagram of MgenDA database. Relational database diagram that contains gene-disease associations and relevant features to aid genomic analysis. Table names are indicated in gray rectangles, with column names on the right and type of information on the left. Relations between tables are depicted by arrows. Database entry identifier depicted by id. AF indicates the allele frequency of the populational study associated with the source of information, whereas maxAF indicates the maximum allele frequency identified from multiple populational studies. DC indicates disease compatibility based on allele frequencies in population studies.

FIG. 3. Diagram of the methodology for the generation of Artificial Neural Networks (ANN) used for text classifications. The top diagram shows a generic representation of the ANN model. Here, N is the number of neurons set as input neurons of the ANN. M and K are the total number of first and second hidden neuron layers. Dashed lines delimit the hidden layers of the ANN. The ANN model is set to have a single output neuron and classification is performed using a sigmoid function on the output values. In the lower diagram, human intervention inputs are highlighted with gray filled oval shapes, rectangles represent the main processes and cylinders represent database structures. The ANN model is generated using a database containing a dataset of extracted text keywords, which have been associated with predefined outcomes for relevant classifications (YES/NO). The methodology systematically generates an ensemble of ANN models with different combinations of hidden neurons, ranging from M=N to 4 in the condition 2<K<M/2. Training and testing of the generated model in the ensemble is automated using a 50% random splitting of the keyword dataset. The ANN model with higher accuracy is selected for deployment. Predictions on new texts with selected ANN models are performed on keywords extracted from the texts. Texts with “positive” outcome are targeted for manual review and evaluation of the text. These will be saved in the database and start a new model generation to update the model and further attempt to improve accuracy.

FIG. 4. Diagram of the automation of the genetic risk assessment on prospective parents (AGDRA). The diagram shows the main steps executed from data from the male and female subjects from their respective databases to the saving of the computed reproductive risk into the patient database. Databases are depicted as cylinders and main processes as rectangles. The order of step execution is depicted numerically next to the processes, where data from male and female subjects are processed in parallel. Male and female symbols indicate data transfers for male and female, respectively.

FIG. 5. Distribution of MIRS values for variants with different classifications. MIRS values were calculated from 24,525 variants with ClinVar classifications. The data was grouped into benign (n=12,827), likely benign (n=5,749), VUS (n=632), likely pathogenic (n=3,520), and pathogenic (n=1,797). VUS indicates Variant of Uncertain Significance, including identified conflicting classifications in the ClinVar database. Variants were selected from the Whole Exome Sequences of 42 cases with revised classifications on the ClinVar database. The data was enriched with 5,291 curated pathogenic/likely pathogenic variants collected from ClinVar that represent distinct genes. MIRS computations were performed using annotations generated using MgenDA and current implementation in its initial version 1.0.

FIG. 6. Examples of variant scoring over a continuous spectrum of MIRS values. Example 1 indicates a case where the MIRS score value is compatible with the range of values within the 95th percentile of benign variants, including likely benign. Example 2 indicates a case where the MIRS score value is compatible with the range of values within the 95^{th} percentile of variants of uncertain significance (VUS), including reported conflicting pathogenicity. Example 3 indicates a case where the MIRS score value is compatible with the range of values within the 95th percentile of pathogenic variants, including likely pathogenic. The 95^{th} percentile estimated using a dataset of 24,525 variants with revised ClinVar database classifications.

FIG. 7. A) Estimated sensitivity and specificity of MIRS as a pathogenic classifier and as a function of cutoff values. B) Receiver Operator Characteristic (ROC) curve of MIRS as a pathogenic variant classifier. Performance metrics were calculated using 24,525 variants containing reviewed ClinVar classifications. For binary classifications, the data was grouped into benign and pathogenic categories. The benign category included all ClinVar classifications with benign and likely benign classifications, whereas conflicting pathogenicity, likely pathogenic and pathogenic were considered for the pathogenic category. True positives were considered when variants belong to the pathogenic group and the MIRS values are above the cutoff value, whereas true negatives are when variants belong to the benign group and the MIRS value is below the cutoff value. When benign cases had MIRS values above the cutoff and pathogenic cases had MIRS values below the cutoff value, these were considered false positives and false negatives, respectively.

FIG. 8. A) Sensitivity of MIRS as a classifier for variant pathogenicity compared with widely used predictive tools. B) Specificity of MIRS as a classifier for variant pathogenicity compared with widely used predictive tools. C) Number of predictions used to estimate the sensitivity and specificity for each classifier in A and B. Performance metrics were calculated using 24,525 variants with classifications reviewed in ClinVar and grouped into benign (negatives) or pathogenic (positives) as described in FIG. 7. A cutoff of 250 was used to apply MIRS as a classifier. Outcomes of 10 widely used predictive tools for accessing the variant impact on gene function (“tolerant” or “deleterious”) as a proxy of pathogenicity¹⁴⁻¹⁶ were collected from multiple in silico studies. “Tolerant” and “deleterious” gene function predictions are considered as “benign” and “pathogenic”, respectively.

FIG. 9. A) Comparison of total reporting times between conventional approach and automated approach using the invention for several work loads. B) Estimated total reporting times of the service using dedicated servers with different numbers of node clusters. Conventional total reporting times were calculated assuming the scenario of 1 working day spent for a bioinformatician to perform WES data processing and 1 working day for a clinical scientist to interpret the resulting candidate variants and generate a final report. Reporting times were calculated for a hypothetical small team (5 bioinformaticians and 5 clinical scientists) and a large team (50 bioinformaticians and 50 clinical scientists). Total reporting times calculated for the “Service” (the invention) were based on an estimated 2.5 h for processing WES data on a single machine on a virtual private server (VPS). Amdahl's Law for distributed computing was applied to calculate the reporting times in the case of 100, 1000 and 5000 computer cluster nodes. Overheads of 0.5 hours with a n×2.5 hours for the serial part of running n jobs in series was used for calculations using Amdahl's Law.

### DETAILED DESCRIPTION OF THE INVENTION

The invention consists of a method for the automated calculation of the reproductive risk of genetic disorders from NGS data of male and female subjects. NGS raw data for the purpose of this invention is generated by established platforms, such as Illumina, Oxford Nanopore Technologies (ONT) and others. In some embodiments, the invention applies to Whole Exome Sequencing (WES) data, Whole Genome Sequencing (WGS) or panels of genes.

The processing, identification, assessment and interpretation of NGS data in this invention has the purpose of identifying pathogenic and/or likely pathogenic genetic variants present in the NGS data of female and male subjects (or egg/sperm providers). Based on the detection of pathogenic or likely pathogenic variants, the invention further provides an automated estimation of the risk of the birth of a future child affected by a life-limiting genetic disease. The invention is designated as a “Single Test” process, meaning the screening of the widest possible spectrum of scientifically known gene-disease associations retrieved from databases and scientific publications.

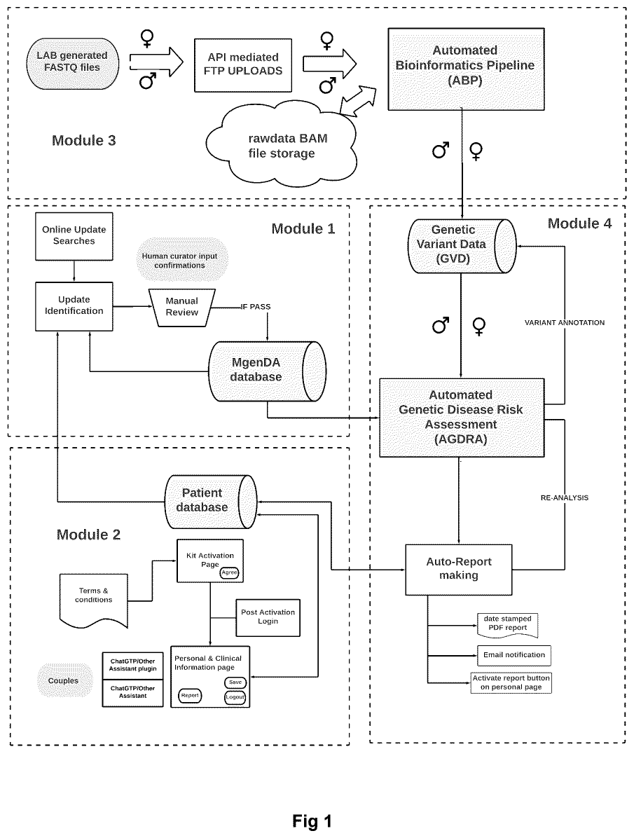

The invention is defined as a system of functionalities (a system as a whole), the “Service”, which provides a fully automated end-to-end data processing framework for a male and female subject (FIG. 1). The Service is composed of 4 core modules, where module 1 is a system database updating functionality and models 2-4 are data-processing modules.

The “Service” starts with the activation and upload of relevant patient data (module 2). The “Service” processes the uploaded genomic data resulting from NGS sequencing, generating the male and female genetic variant data (Module 3). The system further processes genetic variants to identify pathogenic and likely pathogenic variant candidates, computes the genetic risks and generates a digital report (Module 4). The report is then accessible through a user web portal aided by a chatbot that assists with the interpretation of the genomic report, the end of the Service (Module 2).

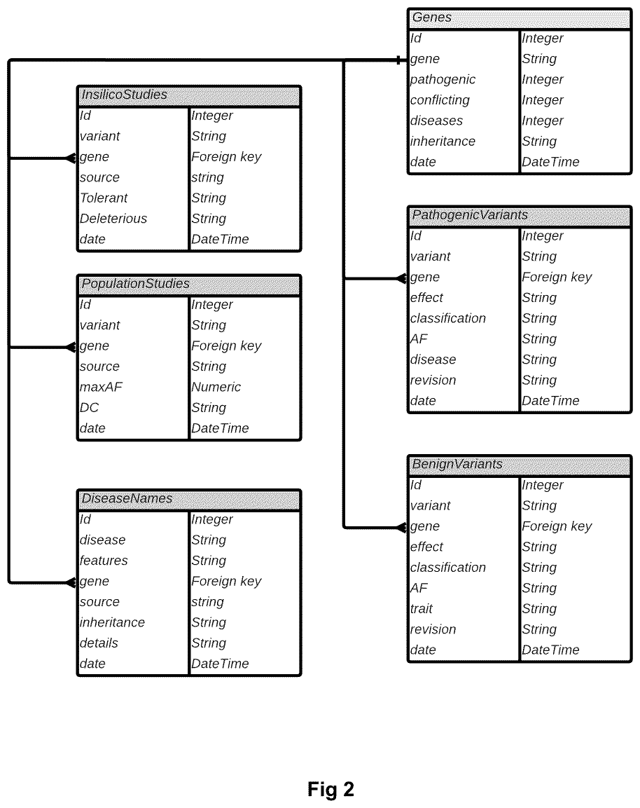

Module 1 ensures the process of curating an internal database for conducting the “Service”. The internal database of this invention consists of an SQL relational database, the MolMart Gene-Disease Association (MgenDA). The database design (FIG. 2) has the purpose of storing in a compact manner the necessary information for conducting the NGS data analysis in module 4.

MgenDA is defined by tables that contain: a) A curated list of genes with known classifications related to disease association (number of pathogenic/conflicting documentation) and its corresponding modes of inheritance. b) List of known gene-disease associations, where the name of the disease and gene are mapped together with the primary source (e.g. OMIM, ClinVar, or other types), inheritance, and details concerning the mechanism of disease; c) Curated list of known pathogenic variants associated with its effect on the gene, the classification status, recommended disease name associated and the allele frequency in the human population; d) A curated list of reported benign variants, the classification status and the allele frequency in the human population; e) List of variants with reported predicted effect on gene function (“tolerant” or “deleterious”) from multiple models collected from multiples in silico studies; f) List of variants with known Allele Frequencies, where maximum and minimum frequencies are collected, alongside disease compatibility inference. Variant identification is tokenized in MgenDA by aggregating the information of chromosome, position, alteration and reference in a single string with “_” separator. This enables an easy and fast querying/matching in data processing steps in module 4. MgenDA tables in FIG. 2 are mainly for illustration purposes as the content may be optimized to meet performance improvements and adapted to other mammalian species.

In module 1, the “Service” database MgenDA is updated with newly reported gene-disease associations and variants detected in well-established databases. These databases include ClinVar, OMIM and other relevant databases. This is achieved by a dedicated API as a “microservice”, programed in a language such as Python or other. The API “microservice” performs systematic online searches for new database releases on the URL repository of the databases, downloads the new version and performs a subsequent comparison and update of the MgenDA database. This process occurs independently of modules 2-4 and enables self-improvement of the database accuracy and gene-disease coverage over time.

In module 1, new database releases are identified by systematic comparison of previous file name releases saved in a log file with dates of download. If a new release is identified, the database is downloaded as tabular text or JSON file through the API connection using a Fast Transfer Protocol. The “microservice” is programed to perform a systematic extraction of the variant information in these files, which is systematically compared with the current MgenDA content to detect putative updates. These comparisons include checking if new information does not exist in MgenDA or if MgenDA contains different information. MgenDA is then updated to include a new variant or to include the new gene-disease association or to modify the table of contents of an existing entry. The current curated MgenDA catalog consists of 25,274 disease names mapped to 393,213 variants from 6,393 different genes, which can be extended over time.

In module 1, the API is programed to perform systematic searches of new relevant scientific publications on PubMed or other scientific database repositories that can potentially enrich MgenDA database content. These searches are conducted according to year of publication to filter several million publications that have been previously evaluated. In each iteration, the PubMed identification numbers are saved in log files to track previous searches and prevent repetition to maximize iteration times. If the PubMed identifier is not in the log file, then the API is programed to fetch the entire text of the abstract.

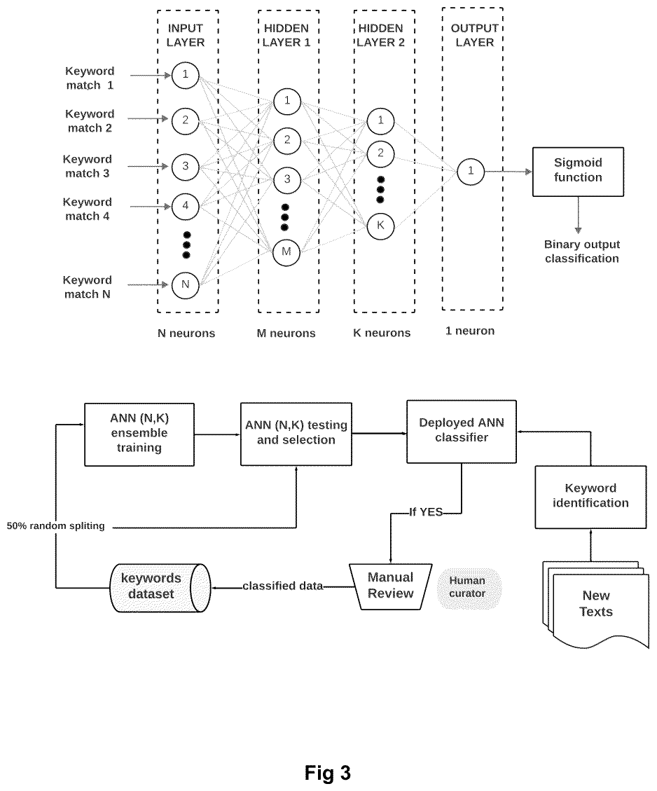

In module 1, extracted Abstracts are classified using a 2-layer Artificial Neural Network (ANN) model trained for the identification of publications reporting new identification of gene-disease associations, including reporting new variants and gene essentiality lists (FIG. 3). The ANN model inputs are a set of keywords (up to 50 words) typically expected to be present in relevant abstracts. By way of example, these keywords are: genomics, NGS, genetic, variant, pathogenic, benign, human, genomics, disease, essential, genes, Inheritance, proband, missense, frameshift, polymorphisms, deletion, “in silico”, patients, infants, and others. The simple ANN output is set as a binary classifier (yes/no) for the relevancy of the abstract using a single output neuron and applying a sigmoid function on the neuron output value. This ANN model design enables the performing of accurate and fast classification of millions of abstracts using less training data and computational power.

In module 1, the text abstract keywords are extracted using Natural Language Processing (NLP) algorithms such as the NLTK in Python. This includes the identification of synonyms using wordnet and other relevant databases or ontologies to capture usage of other words with the same meaning. Existing gene names are included as words mapped with the keyword “gene” to further increase the capacity of identification of a specific gene publication. Different medical terms found in ontologies are also mapped to the word “disease” to further increase the changes to identify new gene-disease associations.

The ANN model in module 1 is achieved by the generation of an ensemble of ANN models with variable number of hidden layers neurons, where a single ANN model with optimal accuracy is selected for deployment according to the diagram in FIG. 3. The data used for model training constitutes a collection of keywords from up to 100,000 abstracts extracted from PubMed. Relevant abstracts are identified by collecting the PubMed IDs identified from the references column of the databases used during the process of MgenDA updating (e.g. ClinVar and OMIM). For the “not relevant” abstracts used in model training, an equal number of PubMed IDs, within the year limit of the last publication considered are randomly generated, excluding any IDs already present on OMIM and ClinVar.

In module 1, a human intervention step is required at the end of the process to ensure the correctness of the automated database updates and suggestions. Database updates are enabled through a manual curation process that checks the alterations identified during the automated online searches and accepts or rejects them. For identified relevant abstracts, this is enabled through reading the publication and evaluating if any information is integrated in the MgenDA database. In the case of finding information relevant to include in MgenDA, the publication reference will be saved as reference in MgenDA and made available to train the ANN model. In the case of not finding any relevant information in the selected publication, the keywords used are saved in the ANN keywords database associated with not relevant abstracts. This starts an automated model re-training event to improve the accuracy of the deployed model, with an aim of improving the accuracy of the ANN model over time.

In some embodiments, the database searches and MgenDA update versions are compatible with the genome reference assembly GRCh38/hg38.

In another embodiment, the database searches and MgenDA update versions are compatible with the genome reference assembly GRCh37/hg19 and other releases of the genome reference assembly.

Module 2 starts the “Service” with the upload of male and female data onto a web portal under user-specific login access (FIG. 1). The data to upload in this module consists of clinically relevant information that includes family history, ethnic background, and familial relationship between the 2 subjects. A step-by-step question-based dialogue with a bot interface is applied at this step to assist the data collection and ensure a bespoke saving of information in the patient database. This is mediated through MolMart Artificial Intelligence Analyst (MAIA). A Large Language Model (LLM) is embedded into the MAIA interface (e.g. ChatGPT or other equivalent framework) to ensure a flexible and scalable interaction with clients. The LLM model is integrated through a plug-in into a bot framework with a pre-programed decision tree of requests to the LLM, using JSON or other formats to communicate with the LLM model. The LLM model is trained using a collection of data that includes customer service scripts, gene-disease specific symptomatology curated internally by genetic counselors. The LLM is controlled by a rule-based decision tree schematic that takes as input the user responses among predefined types of question/answers in a closed loop. This enables a reliable data upload and activation of the service through controlling and limiting the LLM activity in the space of the “Service” purpose. The LLM model plugin is integrated with a graphical interface that contains images, tables and optional clicking choices depending on the context of the conversation to maximize the user experience. Once all patient data is uploaded, a final terms and conditions step is included in the bot conversation framework. If accepted, this initial step enables the upload of genomic data, starting the module 3 data processing. The bot interface is designed to provide users with access to the status of the process, including access to the report generated in module 4. The LLM is trained to perform relevant explanations about the report outcomes and available actionable solutions. This functionality is controlled by the decision tree library of input and output choices available to prevent deviations from the “Service” purpose and unpredictably inaccuracies of responses.

Module 3 of the service consists of the Automation of the standard Bioinformatics Pipelines (ABP) recommended for NGS technologies, following the standard Best Practice Guidelines for clinical applications^{6,13}. These pipelines include sequential steps aided by well-established bioinformatics tools, specific for each type of NGS technology, which can be integrated into this module. In some embodiments, different sequential steps and different tools can be integrated in this module.

In module 3, the input is a FASTQ file on a web portal. The FASTQ file consists of a text-based format for storing nucleotide sequence data and the corresponding quality scores. The “Service” requires the upload of FASTQ files for the male and the female subjects.

The automation element in ABP in this invention is achieved by:

(a) The Implementation of the necessary recommended bioinformatics tools or equivalent as command-line tools in a computer system. This is further controlled by another application (API) as a “microservice” using a script in a language suitable for automation such as Python to run and manage files systematically, including sample acceptance/rejection if failing the pipeline preliminary quality test. System implementation ensures that the generated outputs of each step are the input of the next step in the series. In some embodiments, the invention predicts multiple versions of pipelines that depend on the NGS technology platform (e.g. Illumina, ONT or other), modifications of invention application or a particular case scenario; and alignment with a different reference genome.

(b) Human intervention step that visually inspects quality plots during the FASTQ preprocessing is replaced by a minimum quality filter criterion approach based on cut-off values. QC reports are tabular text files with the laboratory-established metrics for sample quality acceptance/rejection, generated by tools such as FASTQC. The system parses the automated QC reports generated and rejects data when more than 20% of the reads have quality values below 30. This guarantees that more than 80% of the sample sequence has an accuracy of 99.9%. Identification of experimental issues for sample rejection is performed from generated QC reports with the cutoffs of:
5% for adapter contamination,
5% for sequence over-representation,
10% deviations of GC content,
20% sequence duplications
1% Per base N content

The efficiency improvement in the ABP in this invention was achieved using:

a) Uploads of FASTQ files controlled by an application that includes an API as a “microservice” and a Graphical User Interface (GUI). The application connects with the Virtual Private Server where the APB is executed. This will use the Fast Transfer Protocol (FTP) running at the back end. FTP maximizes the upload file transfer speed to 400-800 MBps and is an important feature for scalability as FASTQ files are large (5-15 GB/individual). Testing the maximum load of a couple with 15 GB FASTQ files each, the upload takes approximately 24 minutes on a machine with i7 ×12 cores and with a 50 MB/disk speed. Loading the files into the physical memory of the machine further boosts the file upload, assuming a moderate RAM speed of 20 GB/s and a faster internet connection.

b) FASTQ files are read and all data processing in ABP executed in the physical memory file-based system (RAM) instead of virtual memory (SSD/HDD), and using a suitable queuing system. This is because most tool-processing time is limited by the file reading as the files are large (>5 GB). The total estimated processing time of the current ABP implementation with running of 2 FASTQ files of 15 GB each, took approximately 2 hours running using a i7 ×12 cores machine with a 50 MB/disk speed. Assuming a moderate RAM speed of 20 GB/s, the system improves the processing time up to 400-fold faster with a time scale in the range of seconds. This is compatible with highly scalable processes.

c) An extensive reduction in the annotation conducted by generic tools is an important optimization of the ABP in this invention. This enables saving several hours of computational resources with extensive and unused annotations on 200,000 variants VCFs, using multiple tools such as GATK, ANNOVAR and VEP¹⁰. Furthermore, these can render over 200 additional columns of annotations, which will consume additional storage space. Instead, the ABP annotation only extracts key annotations necessary for the purpose of the invention. These include predicted effects and impacts using robust tools such as SnpEff or other equivalent¹⁰. Other key annotations to extract are the gene name; zygosity; amino acid alterations; HUGO variant nomenclature. Annotations necessary for gene-disease associations are performed in the subsequent module 3 using a faster methodology, which consists in querying MgenDA, enabling average performances of 2 min/case.

d) The final VCF output is converted to an object such as an SQL database or a JSON type of file. This guarantees faster access to information in downstream processes in comparison with common practices of reading and parsing VCFs. Timings are almost instant in the case of the size of an annotated VCF, due to SQL databases and JSON being fast-parsable data structures which are optimized in most programing languages. Performance estimation is in the range of 380-510-fold faster depending on the amount of variants to parse.

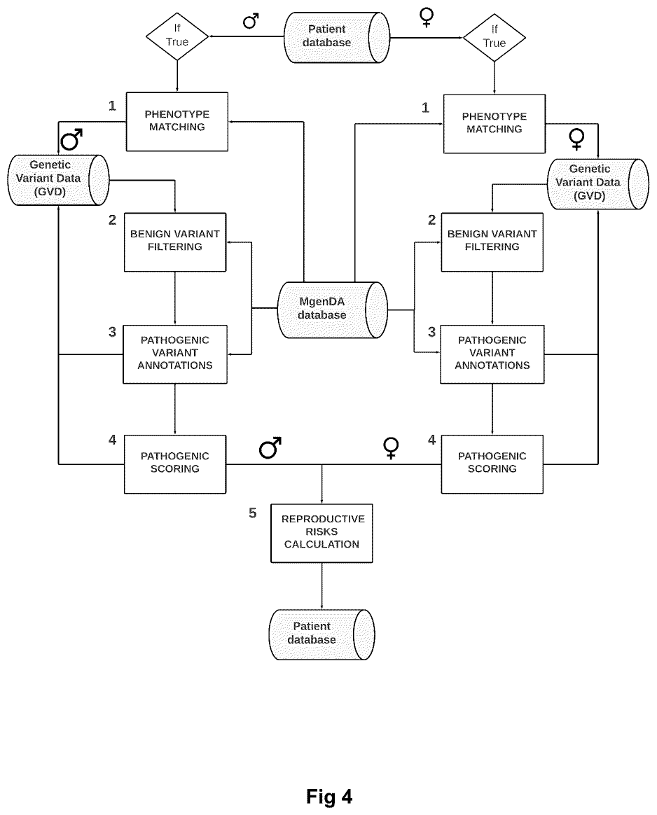

e) Optimal storage of FASTQ as raw data is based on converting into its binary and compact version, the BAM file. This file renders a 2-fold decrease in file size and can be inter-convertible to FASTQ using currently available command line tools in bioinformatics¹⁰. This is fundamental to ensure scalable long-term storage. Module 4 starts with the genetic variants generated for the male and female subjects, saved as a SQL database or JSON file (module 3 outputs). This data is the main input of the Automated Genetic Disorder Risk Assessment (AGDRA), the key process of the invention (FIG. 4). The input data contains all Genetic Variant Data (GVD) identified and is utilized to further store annotations executed during the AGDRA-mediated processing stage.

AGDRA takes as input the text information from the patient database that describes a phenotype, in case of existing information. The inputs are first extracted as fast-parsable objects and the data for male and female subjects is processed in parallel, where the following steps are executed in series:

AGDRA PROCESSING STEP 1, THE PHENOTYPE MATCH. This step identifies genes associated with the provided family history of the male and female subjects. In a different embodiment, it identifies gene(s) associated with the provided phenotype of the male subject in the case of suspected autosomal dominant disease. In a different embodiment, it identifies gene(s) associated with the provided phenotype of the female subject in the case of suspected autosomal dominant disease.

Step 1 of AGDRA takes as input the text description of the genetic disease (Phenotype of the subject; or Family History). If the phenotype description is absent, this step is omitted. The input text description is systematically matched with the MgenDA database records of disease names. This is based on a computed score that calculates the percentage of keywords in disease names that are associated with extracted keywords from the text description executed. The scoring strategy enables the selection based on a % cut-off that can be optimized to maximize the difference between true positives and false positives using machine learning techniques on real patient data.

Keywords are systematically matched by combining NLP algorithms such as the Python NLTK method with the fuzzy pattern matching such as the Python fuzzywuzzy method. Databases of medical ontologies and terms are considered to ensure synonyms matches are possible. Once the disease name match is selected, a programed database query on MgenDA to get the set of genes associated is executed. The performance of step 1 in its current implementation rendered a Sensitivity of 91.7% with a False Discovery Rate of 5.7%, tested on 433 texts generated with disease descriptions with all the keywords from randomly selected disease names from the MgenDA database.

AGDRA PROCESSING STEP 2, BENIGN VARIANT FILTERING. The purpose of this step is to remove benign gene variants as candidates. This step takes as input the fast-parsable objects containing the gene variants of male and female subjects. MgenDA is used to identify the relevance of the candidates by querying its tables with variant identifiers, using the tokenized forms that contain chromosome, position, alteration and reference. Filtering benign variants removes about 96% of data which contains variants of no clinical significance before executing extensive annotations and database querying. This step enables an improvement in computational efficiency as it substantially minimizes the number of database querying events.

In AGDRA step 2, the first filter is executed by identifying disease-incompatible variants to discard using the MgenDA table of Population Studies¹⁷. The invention uses the maximum Allele frequency of the variant to infer its incompatibility with disease. With current data, an estimated cutoff value of >0.3 is assumed as incompatible with pathogenicity. The value was estimated by identifying the minimum value within the 95th percentile of reported pathogenic variants from the ClinVar database. In some embodiments, modifications of the cutoff value are applied to improve “Service” performance. This approach is panethnic which enables our invention to be applied to ethnically diverse populations.

In AGDRA step 2, a second filter is applied to check that the variant is in a gene associated with a disease. In a different embodiment of this step, the second filter is applied to check that the variant belongs to an essential gene.

In AGDRA step 2, a third filter is applied to check if the variant is contained in a list of benign variants present in the MgenDA database. This enables further discarding of variants with low allele frequency or absent in population studies. The resulting candidates from AGDRA step 2 are marked and saved in the GVD which is a SQL database/JSON file. This procedure further saves computational storage resources and enables tracking of the “Service” process.

AGDRA PROCESSING STEP 3, PATHOGENIC VARIANT ANNOTATIONS. The purpose of this step is to collect evidence of pathogenicity for gene variant candidates from male and female subjects. The input is the two lists of variants from Step 2. MgenDA is queried to execute the key annotations necessary for the automated identification of pathogenic, likely pathogenic and conflicting pathogenicity variants. Tokenized variant identification that contains chromosome, position, alteration and reference is used to query MgenDA for these annotations. For each variant, MgenDA is queried for:
a) Associate the known mode of inheritance with the genetic disorder. This can be either autosomal, X-linked or Y-linked types in the forms of dominant or recessive. In another embodiment, predisposition to a disease is taken into account;
b) The pathogenic classification obtained from the curated version of ClinVar database and the correspondent review status. These would include Pathogenic, Likely Pathogenic and Conflicting Pathogenicity;
c) Inference of Allele Frequencies identified in population studies into “degrees compatible” and “incompatible” with genetic disease;
d) Essentiality of a gene. This includes the process by which this gene is essential;
e) Predicted impact on the loss of gene function (“tolerant” or “deleterious”) from in silico studies from multiple models/tools (e.g. SIFT, Polyphen2, LRT, Mutation Taster, MutationAssessor, FATHMM, MetaSVM, MetaLR, PROVEAN′, M-CAP, fathmm-MKL, and others).

In AGDRA step 3, the generated annotations in the variants in combination with the ones provided in the output of ABP (module 1), are used to execute an automated inference of the evidence of pathogenicity according to ACMG guidelines or other professional Best Practice Guidelines^{11,12}. For example, the following ACMG criteria is applicable to asymptomatic individuals:
PVS1, evidence from null variant with high impact (Very Strong).
PS1, evidence from pathogenic missense variant with same aa alteration (Strong)·
PM2, low frequency in populations compatible with rare diseases (Moderate)·
PM4, inframe deletions insertions and stop loss (Moderate)·
PM5, Novel aa missense (Moderate)·
PP3, Evidence from multiple in silico models (Supporting)·
PP5, Evidence from databases (Supporting)

Based on the resulting annotated evidence from AGDRA step 3, the classification of pathogenicity (pathogenic, likely pathogenic or VUS) is calculated according to ACMG criteria or other professional Best Practice Guidelines^{11,12}. After all annotations are executed, variant candidates are selected based on a minimal evidence criterion. The selection is made if one of the following is verified:
The variant is reported as pathogenic/likely pathogenic in ClinVar database.
The variant is predicted to affect the loss of function of a gene associated with a genetic disease.
The variant is classified as pathogenic/likely pathogenic based on a combination of ACMG criteria or other professional Best Practice Guidelines.

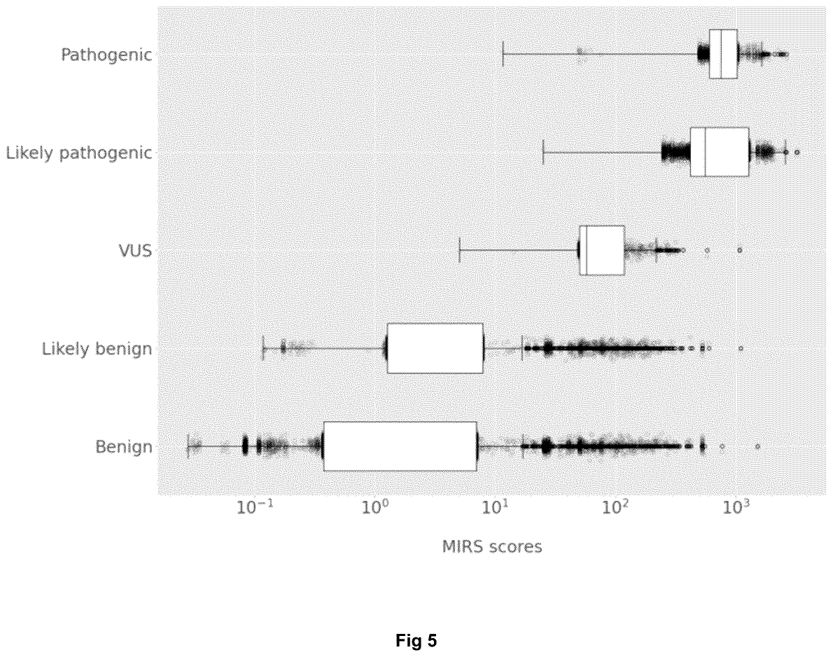

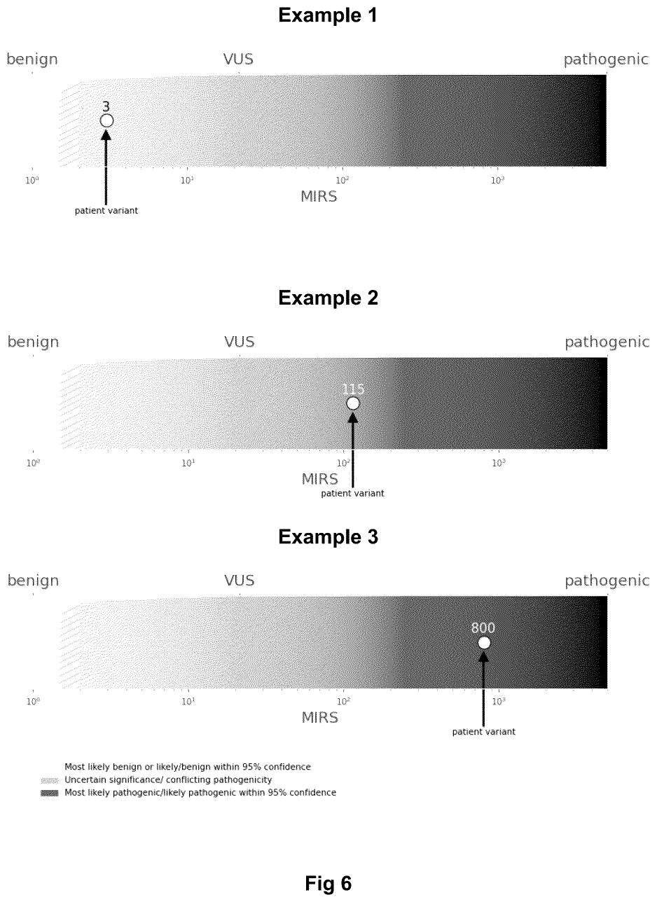

AGDRA PROCESSING STEP 4, COMPUTING PATHOGENIC SCORES. The purpose of this step is to compute a score that reflects the degree of pathogenicity of a variant. The input is the male and female candidate variants in the GVD resulting from AGDRA step 3. The scoring system present in this invention is the MolMart Integrative Ranking Score (MIRS). The system is defined as a quantitative metric that weights multiple sources of pathogenic evidence used in genomic analysis to evaluate and decide on the pathogenicity potential of a variant. In some embodiments, other scoring systems, variations of MIRS parameters, or extensions of its variables are applied to increase the “Service” performance. The MIRS system used in this invention is novel and was derived mathematically to enable discrimination between benign, uncertain and pathogenic variants which fall into distinct orders of magnitude (FIG. 5 and FIG. 6). MIRS is calculated by the following equation:

$$MIRS = P ( I \frac{1}{m} {\sum }_{i}^{m} Si + D + A + F ) \qquad equation 1$$

Where:
P is a correction factor that relates to the compatibility with having a disease based on the known allele frequencies from population studies. Numerically, this is a factor that can reduce an order of magnitude of the overall MIRS score. For example, in the current implementation, it takes the value 0.1 for variants with allele frequencies incompatible with disease and the value 1 for compatible. In some embodiments, the values can change and adopt multilevel correlation factors.
I is the weight for the predicted impact of the variant on the gene function. Multiple degrees of impact with discrete levels within 2 orders of magnitude are considered. For example, predicted impacts annotated from tools such as SnpEff resulted in the outcomes of “HIGH”, “MODIFIER”, “MODERATE” and “LOW” impacts. The automated “Service” associates values of 100, 25, 10 and 1 to such outcomes.
Si is the weight from the predicted effect on gene function from in silico studies using the model i of m models. Values can range from 0 to 100 depending on the number of in silico studies with different datasets available and their outcomes (“tolerant” or “deleterious”). Tolerant is associated with the minimum value, whereas deleterious to the maximum value.
D is the weight for the reported variant classification in well-established databases (e.g. ClinVar or equivalent). Pathogenic classification is associated with the maximum value, whereas benign classification is associated with the minimum value. Intermediate values are associated with other classifications where D (“likely pathogenic”)>D (“VUS/conflicting”)>D (“likely benign”). Current optimal values are estimated to be 500, 250, 50, 1 and 0.1 for Pathogenic, Likely Pathogenic, VUS, Likely Benign and Benign, respectively.
A is the weight for the computed ACMG criteria for the classification of a variant. Pathogenic classification is associated with the maximum value, whereas benign classification is associated with the minimum value. Intermediate values are associated with other classifications where A (“likely pathogenic”)>A (“VUS/conflicting”)>A (“likely benign”). Current optimal values are estimated to be 1000, 500, 0, −1 and −10 for Pathogenic, Likely Pathogenic, VUS, Likely Benign and Benign, respectively.
F is the weight associated with the patient family history of genetic diseases. F takes values from 0-100 depending on if there is a gene match in step 1 of AGDRA and the relative's proximity to the patient where the disease is observed. For example, the maximum value is associated if the disease is observed in the patient and the gene has a phenotype match. The value 0 is associated when there is no disease in the family history or no gene match. Intermediate values are associated with the 100/n where n is the degree of family proximity.

In step 4 of AGDRA, Si is calculated respectively from the following equations:

$$Si = {\sum }_{j}^{n} Mij \frac{1}{n + k} \qquad equation 2$$

Where:
Mij is the weight of the variant predicted effect on the gene function by the model i in the in silico study j. The weight can take values from 0-100, depending if the prediction is deleterious (max value), intermediate or tolerant (“minimum value”).
n is the number of in silico predictions in the MgenDA database from different studies or using different datasets.
k is a coefficient for the number of positive studies that give a weight of ½ of the maximum value. The value was estimated by fitting it to data using machine learning techniques.

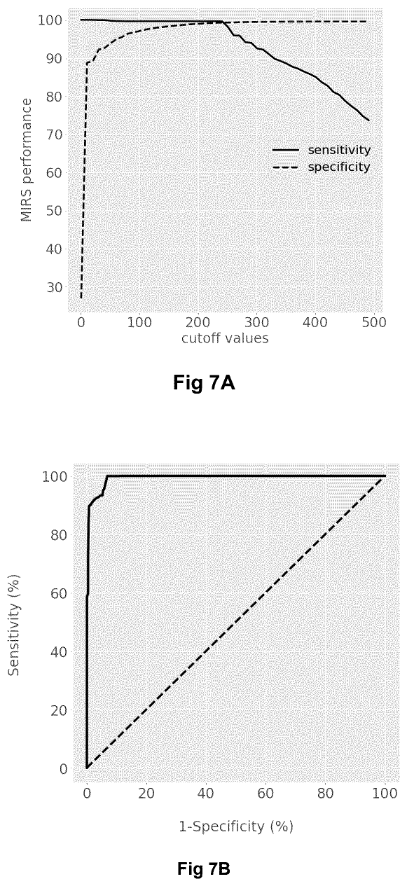

AGDRA PROCESSING STEP 5, COMPUTING REPRODUCTIVE RISKS. The purpose of this step is to identify genetic diseases that can be passed to the offspring from the prospective sperm/egg donors' genetic variants. This step takes as inputs the variants and their annotations, including the computed MIRS, obtained from step 4 of AGDRA. This includes the data from male and female subjects in the GVD. Step 5 performs a systematic gene-by-gene search on variants from male and female subjects to find variants with significant MIRS values in the same gene (a “gene match”). At this step, a MIRS cutoff value is applied to rule out remaining benign/likely benign variants and consider variants with pathogenic/likely pathogenic classification. The MIRS cutoff value is estimated from large datasets of variants with revised pathogenic classifications to ensure optimal performance of the “Service” (FIG. 7A). In some embodiments, VUS are considered in the “gene match” requiring fine-tuning of the cutoff value. A “gene match” is accepted if a variant in the data from the male subject with MIRS higher than the cutoff and a variant in the data from the female subject with a MIRS higher than the cutoff are identified in the same gene. If a “gene match” occurs, the genetic variant and its associated information are targeted to compute the reproductive risk. The methodology enables the “Service” to cover any possible scenario of variants that cause loss of gene function, regardless of the variant similarity between male and female or its location in the gene. Variants with MIRS higher than the cutoff and no “gene match” are selected if the following conditions are verified: a) Annotated evidence of autosomal dominance, consisting of checking if the gene has been previously matched with the reported family history (AGDRA step 1) or is compatible with a highly variable penetrance. b) The variant is located on the X chromosome of the female subject and the gene is associated with a disease in MgenDA. This enables the “Service” to detect X-linked recessive diseases, c) The variant is located on the Y chromosome of the male subject and is compatible with the male phenotype or has highly variable penetrance.

In AGDRA step 5, the “Service” automatically computes the male and female combined reproductive risk for each gene using the selected gene variants and based on their annotations. Reproductive risks are computed by applying Mendelian law. The risks are computed in conditions compatible with family history and zygosity, including variants in the same gene whose combined presence results in compound heterozygosity. This enables the invention to discard candidates that have no disease penetrance in an automated manner (False positives). The application of Mendelian law is illustrated in the following main case scenarios:
a Autosomal recessive inheritance with asymptomatic progenitors. This applies in the case of a “gene match” in step 5 of AGDRA. The risk is calculated as 1 in 4 if both male and female zygosity is annotated as heterozygous.
b X-linked recessive inheritance with asymptomatic female subject. This applies when a pathogenic/likely pathogenic variant is identified in the female and not in the male. The risk is calculated as 1 in 2 if the gender of the resulting embryo is male and 0 if female.
c Dominant cases. These include the identification of pathogenic/likely pathogenic variants in any chromosome, regardless of the “gene match” of variants.
d Gender of the offspring is considered for the risk calculation, if the variant is associated with an X-linked or Y-linked disorder.

In step 5 of AGDRA, the resulting genes with computed risks are associated with the recommended disease names annotated from the MgenDA database. In some embodiments of the service, the computed risks are associated with genes essential for key biological processes or other relevant traits. Resulting reproductive risks, genes, variant identifiers and associated diseases/essential processes/traits are saved in the patient database.

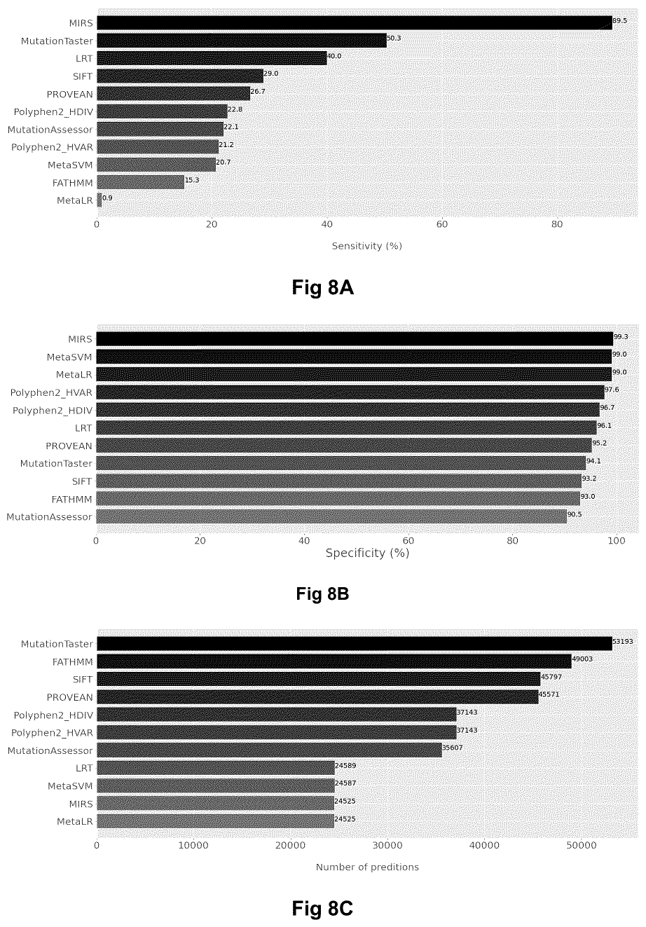

The AGDRA processing unit was tested by using artificially generated variant data from egg/sperm donors rendering a dataset of 5,291 different cases. Each case was designed to contain one pathogenic/likely pathogenic variant to be detected among a total of 110,000 variants (average number of exome variants in humans). In this testing dataset, pathogenic/likely pathogenic variants were randomly selected from the ClinVar database to generate a representative variant for each gene that is associated with a known genetic disease. These included autosomal recessive, autosomal dominant and X-linked cases. The remaining variants were generated by randomly selecting from a pool of ˜700.000 variants from real cases, which did not contain pathogenic/likely pathogenic classified variants in ClinVar. Current implementation obtained a 97% accuracy in detecting the genetic disease using the default MIRS cutoff of 250. In addition, this implementation has a high predictive power for pathogenic variant identification (FIG. 7B) with estimated sensitivity and specificity gains superior to the usage of commonly used predictive tools (FIG. 8) 14-16 The invention enables further fine-tuning the accuracy of the method by altering the cut-off values which alters the performance of pathogenic/likely pathogenic detection by the scoring system (FIG. 7A).

In module 2 of the “Service”, the Auto-Report function consists of a control unit that automatically generates a digital genomic report after the AGDRA computations are executed. The unit consists of a program that acts as an independent “microservice” and automatically populates a standard genomic report template with relevant patient information and results from step 5 of AGDRA. The genomic report is designed to contain information regarding the calculated reproductive risk, associated disease name or phenotype, pathogenic gene variants identified and recommended actionable solutions. The layout of the template is designed to contain written, table and figure contents to facilitate the user's understanding, for example, figures showing the obtained pathogenic score for reported variants (FIG. 6).

The unit is connected with the patient database, where the necessary patient information is retrieved for report generation and the AGDRA output results are saved. This unit controls the AGDRA process, enabling optional re-analysis of the data if requested by the user through module 2 interface. The unit executes date stamping of report generation, email notification to users and activates visualization/download functionally on the user's personal page. Reports are saved in Portable Document Format (PDF) or other digital format. These functionalities are mediated through the patient database connection with module 2 interface.

The integration of the sum of the system functionalities of the invention constitutes the scalable “Service”, which addresses the limitations described in the background. Based on these functionalities, the “Service” can provide the screening of all known genetic disorders in a single test. The estimated overall turnover of the “Service” is about 2 h 30 min for WES data of egg/sperm donors uploaded in module 3 using a 12 core 2.8 GHz VPS machine. This automates the process by removing some of the labor-intensive steps found in routine practice in clinical bioinformatics for genomic data processing, candidate variant interpretation and report drafting and represents a cost-efficient and scalable alternative to current practice^{7,11,12}.

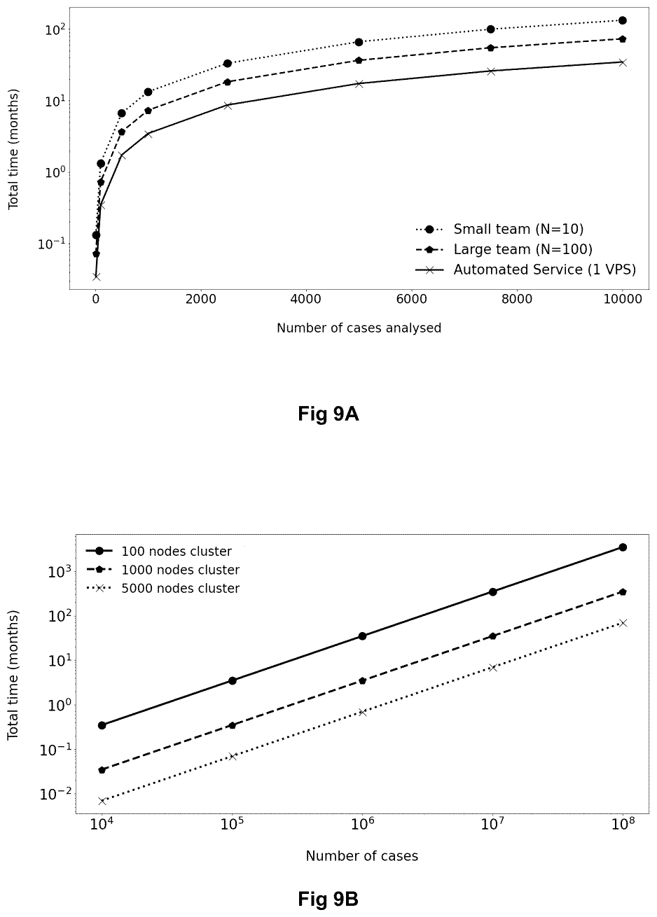

The invention outperforms by a factor of 2 to 4-fold the conventional genomics laboratory protocol approach assuming a team of 100 people (FIG. 9A). By integrating the invention into a computer cluster with multiple nodes, the technology can be scaled by multiple orders of magnitude and applied for population-wide screening (FIG. 9B).

### REFERENCES

1. The Lancet Global Health. The landscape for rare diseases in 2024. Lancet Glob. Heal. 12, e341 (2024).
2. Yang, G. et al. The national economic burden of rare disease in the United States in 2019. Orphanet J. Rare Dis. 17, 163 (2022).
3. Lichstein, J. et al. Children with genetic conditions in the United States: Prevalence estimates from the 2016-2017 National Survey of Children's Health. Genet. Med. 24, 170-178 (2022).
4. Miller, K. E. et al. The Financial Impact of Genetic Diseases in a Pediatric Accountable Care Organization. Front. Public Heal. 8, 1-9 (2020).
5. Boonsawat, P. et al. Assessing clinical utility of preconception expanded carrier screening regarding residual risk for neurodevelopmental disorders. npj Genomic Med. 7, 45 (2022).
6. Fresard, L. & Montgomery, S. B. Diagnosing rare diseases after the exome. Mol. Case Stud. 4, a003392 (2018).
7. Koboldt, D. C. Best practices for variant calling in clinical sequencing. Genome Med. 12, 91 (2020).
8. Schuler, B. A. et al. Lessons learned: next-generation sequencing applied to undiagnosed genetic diseases. J. Clin. Invest. 132, 1-9 (2022).
9. Roman-Naranjo, P., Parra-Perez, A. M. & Lopez-Escamez, J. A. A systematic review on machine learning approaches in the diagnosis and prognosis of rare genetic diseases. J. Biomed. Inform. 143, 104429 (2023).
10. Pereira, R., Oliveira, J. & Sousa, M. Bioinformatics and computational tools for next-generation sequencing analysis in clinical genetics. J. Clin. Med. 9, (2020).
11. Karczewski, K. et al. ACGS Best Practice Guidelines for Variant Classification in Rare Disease 2020. bioRxiv 531210 (2019).
12. Richards, S. et al. Standards and guidelines for the interpretation of sequence variants: A joint consensus recommendation of the American College of Medical Genetics and Genomics and the Association for Molecular Pathology. Genet. Med. 17, 405-424 (2015).
13. Pirooznia, M. et al. Validation and assessment of variant calling pipelines for next-generation sequencing. Hum. Genomics 8, 14 (2014).
14. Montenegro, L. R., Lerario, A. M., Nishi, M. Y., Jorge, A. A. L. & Mendonca, B. B. Performance of mutation pathogenicity prediction tools on missense variants associated with 46,XY differences of sex development. Clinics 76, e2052 (2021).
15. Dong, C. et al. Comparison and integration of deleteriousness prediction methods for nonsynonymous SNVs in whole exome sequencing studies. Hum. Mol. Genet. 24, 2125-2137 (2015).
16. Cannon, S., Williams, M., Gunning, A. C. & Wright, C. F. Evaluation of in silico pathogenicity prediction tools for the classification of small in-frame indels. BMC Med. Genomics 16, 1-9 (2023).
17. Walsh, R., Tadros, R. & Bezzina, C. R. When genetic burden reaches threshold. Eur. Heart J. 41, 3849-3855 (2020).

1) A novel method for automating genetic disease risk assessment, comprising of:
i) systematic identification of pathogenic genetic variants from genomic data of a male and a female subject;
ii) based on (i), systematic pairing of pathogenic variants from a male and a female subject to calculate reproductive risk in offspring for each gene, where there is a gene-disease association;
iii) for each male and female subject analyzed in (i) and (ii), matching of user-provided text, relating to the subjects' phenotype and family history, with genetic disorder names to identify relevant genes;
iv) based on (i), (ii) and (iii), calculating reproductive risk for genes associated with Autosomal Recessive Mendelian inheritance; Autosomal Dominant Mendelian inheritance; X-linked Mendelian inheritance; Y-linked Mendelian inheritance,
wherein the method in (1) is configured as a fully automated end-to-end process (a system as a whole), enabling the scaling of calculating reproductive genetic risk by solving the limitations of current methodologies; and wherein the method in (1) enables the application of the method to non-symptomatic individuals as a preventative genetic screening tool and deployment as a population screening tool; and wherein the method in (1) is applicable to humans and other mammalian species.

2) A novel method for a continuous scoring system of pathogenicity for genetic variant classification, described as MolMart Integrative Ranking Scoring (MIRS), comprising of a metric system that uses numerical weights in different scales to combine evidence of pathogenicity from: existing variant classifications in databases; inferred variant classification based on well-established publishes criteria; frequency-based observations compatible with pathogenicity; predicted impact on gene functionality; predicted loss of gene functionality from published in silico studies using diverse tools; pathogenic phenotype observations in family history.
The method in (2) provides the means to discriminate between benign and pathogenic variants in a continuous scale, describing intermediate classifications such as likely benign, Variant of Uncertain Significance (VUS) and likely pathogenic. The methodology enables accurate pathogenic classification of genetic variants and is used in claim 1), wherein the process augments the performance of pathogenic variant identification in the field by integrating multiple sources of pathogenic supporting evidence and is applicable to pathogenic classification of genetic variants in humans and other mammalian species.

3) A novel method for deploying and updating a proprietary database (MolMart Gene Disease Association-MgenDA) containing gene-disease associations, comprising of:
i) compact design for efficient variant annotation, containing information to facilitate pathogenicity scoring and gene-disease association;
ii) based on the existing version of (i), systematic updating of MgenDA content by performing automated integrations of new database releases from the corresponding online repositories;
iii) based on the existing version as an output of (i) and (ii), systematic identification of scientific publications relating to discovery of new genetic pathogenicity, by means of an Artificial Neural Network (ANN), with the purpose of integration into MgenDA
iv) automated training and selection of the ANN model in step (iii) for improving the accuracy of abstract identification, based on the feedback data from the curator;
The method according to claim 3) is used to perform steps (i) to (v) of the method in claim 1,
wherein the method in (3) improves the accuracy of database-driven genetic findings and increases the number of gene-disease or gene-trait associations over time. And wherein the method in (3), facilitates the early corporation of new findings into MgenDA before they become incorporated into mainstream databases.

4) A process for efficient automation of NGS data processing and analysis, in the context of reporting reproductive genetic risk, according to the method in claim 1) for the system as a whole, comprising of:
i) automation of standard bioinformatics pipelines for NGS data processing controlled by an API as a standalone microservice;
ii) dependent on (i), automated NGS quality control assessment based on multiple threshold cutoffs for sample acceptance/rejection;
iii) based on the output of (i) and (ii), an optimized system for FASTQ data upload, file storage in BAM format and memory-base file reading;
iv) variant annotations and risk calculations performed by the method in claim (1), using a compact fast-querying database (MgenDA), described in claim 3);
v) an independent microservice for the automated population of genomic digital reports, dependent on the output of step (iv);
wherein the process in (4) is configured to optimize performance and scalability by reducing data retrieval and processing times. And wherein the process in (4) is applicable to the classification of genomic variants in humans and mammalian species.

5) A novel method for facilitating genomic data collection and result interpretation online, according to the method in claims 1) and (4), comprising of:
i) a proprietary plug-in with a trained LLM for mediating the user interaction in the context of the “Service” activation, family history data collection, genetic report generation and post-result counseling based on the input of claim 4) and the output of claim 1);
ii) dependent on the input and output of (i), a bot framework with a graphical user interface in a web portal, under the proprietary name MolMart Artificial Intelligence Analyst (MAIA) that mediates the user interaction;
wherein the method in claim 5) enables the scalability of the “Service” with an effective result interpretation follow-up, by reducing reliance on human-led steps. And wherein the method in claim 5) constitutes a novel application of the OpenAI framework.

6) Use of the method, according to any of the claims 1 to 5 for the identification and interpretation of reproductive risk in a male and a female subject or any pairwise combination of male and female subjects in humans or other mammalian species.

7) Use of the method, according to any of the claims 1 to 5 for the identification and interpretation of specific genetic traits in a male and a female subject or any pairwise combination of male and female subjects in humans or other mammalian species.
